(19) United States
(12) Patent Application Publication
(10) Pub. No.: US 2004/0198763 A1
(43) Pub. Date: Oct. 7, 2004

(54) Method of treating dry eye with a macrolide compound

(75) Inventor: Ryuji Ueno, Potomac, MD (US)

Correspondence Address: OBLON, SPIVAK, MCCLELLAND, MAIER & NEUSTADT, P.C., 1940 DUKE STREET, ALEXANDRIA, VA, 22314, US

(73) Assignees: Sucampo AG, Zug (CH); Sucampo Pharmaceuticals, Inc., Bethesda, MD

(21) Appl. No.: 10/758,260

(22) Filed: Jan. 16, 2004

Related U.S. Application Data
(60) Provisional application No. 60/440,388, filed on Jan. 16, 2003.

(51) Int. Cl. A61K 31/4745

(52) U.S. Cl. 514/291

(57) ABSTRACT

The invention provides methods of treating dry eye, which entail administering an effective amount of a macrolide compound, such as tacrolimus, to a patient having a Schirmer score of less than or equal to seven millimeters per five minutes and/or an SPK score of at least two.

### TECHNICAL FIELD OF THE INVENTION

The present invention relates to a method for treating dry eye.

### BACKGROUND OF THE INVENTION

Dry eye syndrome (keratoconjunctivitis sicca) is generally characterized by a deficiency in tear production and/or tear composition that may result from a variety of toxic or inflammatory insults. In many cases, no specific cause can be identified, but it typically is associated with considerable discomfort. It often results in protracted inflammation of the outer covering of the eye and can result in impaired sight or blindness. Aging, eye stress from reading or computer work, a dry environment and using various medications appear to be the main causes of dry eye. Women report more dry eye than men.

While dry eye can occur at any age, it is primarily a disorder of the elderly. Some estimates suggest that 75% of those over 65 years will experience dry eye. In the younger population, it has been suggested that the cause of dry eye is of a secondary, evaporative nature associated with contact lens wear. Current estimates indicate that over 12 million people in the United States suffer from dry eye syndrome.

Based on the severity of the disease, dry eye can be categorized as mild, moderate and severe. In the United States, moderate-to-severe dry eye affects approximately 4.2 million people. The prevalence of dry eye is expected to increase due to the aging of the US population.

Dry-eye treatment may vary depending on the severity of the condition. The most common methods of treating dry eye are the use of artificial tears and lubricating ointments. However, these current treatments only provide temporary relief of dry-eye symptoms and are inadequate for patients suffering with moderate-to-severe dry eye.

With so many patients suffering from dry eye, and with no cure for the condition, there is a large unmet medical need for new therapeutic agents and treatment regimens for dry eye.

### SUMMARY OF THE INVENTION

It is, therefore, an object of the present invention to provide useful, improved compositions and methods for treating dry eye syndrome with macrolide compounds.

Another object of the present invention is to provide a commercial package comprising the composition of the present invention and a written matter associated therewith, the written matter stating that the composition can or should be used for dry eye.

According to this and other objects of the invention, a method of treating a human patient suffering from dry eye is provided. According to one embodiment, this method entails administering to the patient an ophthalmic composition containing from about 0.01% to about 0.1% of a macrolide compound. In other embodiments, the method involves administering to the patient an ophthalmic composition containing from about 0.03% to about 0.06% of a macrolide compound, preferably about 0.03%.

In still other embodiments, the target patient population has a Schirmer score of less than 7, preferably less than 5, millimeters per five minutes. In other embodiments, the patients have a superficial punctate keratitis score of greater than or equal to 2, preferably greater than or equal to 3.

Preferred compositions are formulated as eye drops, which optionally contain polyvinyl alcohol, or ointments. In general, these compositions will be administered to the eye from about one to about four times per day.

Preferred macrolide compound is a tricyclo compound having the following formula (I) or a pharmaceutically acceptable salt thereof:

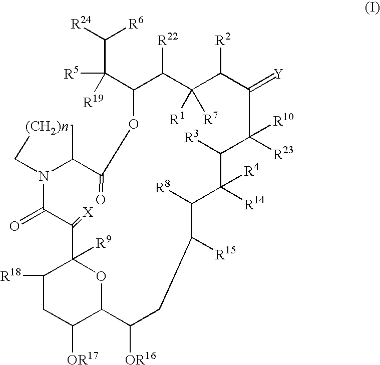

wherein adjacent pairs of R¹ and R², R³ and R⁴, and R⁵ and R⁶ each independently: (a) consist of two adjacent hydrogen atoms, wherein R² is optionally alkyl, or (b) form another bond optionally between carbon atoms binding with the members of said pairs; R⁷ is hydrogen atom, hydroxy, alkyloxy or protected. hydroxy, or may form oxo with R¹; R⁸ and R⁹ each independently show hydrogen atom or hydroxy; R¹⁰ is hydrogen atom, alkyl, alkyl substituted by one or more hydroxy, alkenyl, alkenyl substituted by one or more hydroxy or alkyl substituted by oxo; X is oxo, (hydrogen atom, hydroxy), (hydrogen atom, hydrogen atom), or a group of the formula —CH₂O—; Y is oxo, (hydrogen atom, hydroxy), (hydrogen atom, hydrogen atom), or a group of the formula N—NR¹¹R¹² or N—OR¹³; R¹¹ and R¹² each independently show hydrogen atom, alkyl, aryl or tosyl; R¹³, R¹⁴, R¹⁵, R¹⁶, R¹⁷, R¹⁸, R¹⁹, R²² and R²³ each independently show hydrogen atom or alkyl; R²⁴ is an optionally substituted ring that may contain one or more hetero atom(s); and n is 1 or 2. Tacrolimus is most preferred.

### DETAILED DESCRIPTION OF THE INVENTION

The present inventor has surprisingly discovered that certain macrolide compounds can be used in specific concentration ranges to treat the dry eye syndrome and symptoms associated with dry eye. In particular, macrolide compounds like FK506 (tacrolimus), ascomycin, rapamycin and their derivatives, can be used in concentrations ranging from about 0.01% to about 0.1% in ophthalmic compositions to treat dry eye syndrome and symptoms associated with it. Moreover, the present inventor has discovered that patients having Schirmer scores less than about 7 to less than about 5 millimeters per five minutes and/or superficial punctate keratitis scores of greater than or equal to 2 or greater than or equal to 3 respond particularly well to treatment.

Macrolide Compounds of the Invention

A specific example of a macrolide compound usable in the invention is a tricyclo compound as shown by the following general formula (I) or a pharmaceutically acceptable salt thereof:

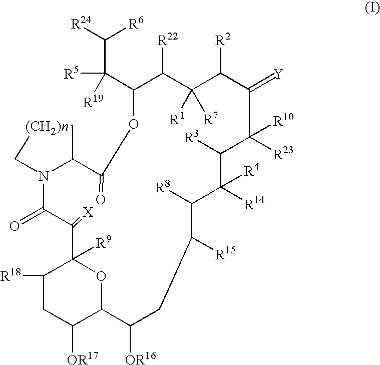

wherein adjacent pairs of R¹ and R², R³ and R⁴, and R⁵ and R⁶ each independently

a) consist of two adjacent hydrogen atoms, wherein R² is optionally alkyl, or

b) form another bond optionally between carbon atoms binding with the members of said pairs;

R⁷ is hydrogen atom, hydroxy, alkyloxy or protected hydroxy, or may form oxo with R¹;

R⁸ and R⁹ each independently show hydrogen atom or hydroxy;

R¹⁰ is hydrogen atom, alkyl, alkyl substituted by one or more hydroxy, alkenyl, alkenyl substituted by one or more hydroxy or alkyl substituted by oxo;

X is oxo, (hydrogen atom, hydroxy), (hydrogen atom, hydrogen atom), or a group of the formula —CH₂O—;

Y is oxo, (hydrogen atom, hydroxy), (hydrogen atom, hydrogen atom), or a group of the formula N—NR¹¹R¹² or N—OR¹³;

R¹¹ and R¹² each independently show hydrogen atom, alkyl, aryl or tosyl;

R¹³, R¹⁴, R¹⁵ R¹⁶, R¹⁷, R¹⁸, R¹⁹, R²² and R²³ each independently show hydrogen atom or alkyl;

R²⁴ is an optionally substituted ring that may contain one or more hetero atom(s); and

n is 1 or 2.

In addition to the meaning noted above, Y, R¹⁰ and R²³ may show, together with the carbon atom they bind with, a saturated or unsaturated 5 or 6-membered heterocyclic group containing nitrogen atom, sulfur atom and/or oxygen atom, the heterocyclic group being optionally substituted by one or more group(s) selected from alkyl, hydroxy, alkyloxy, benzyl, a group of the formula —CH₂Se(C₆H₅), and alkyl substituted by one or more hydroxy, or its pharmaceutically acceptable salt.

In the general formula (I), preferably R²⁴ is, for example, cyclo(C₅-C₇)alkyl optionally having suitable substituent, such as the following.

(a) 3,4-dioxocyclohexyl

(b) 3-R²⁰-4-R²¹-cyclohexyl,

wherein R²⁰ is hydroxy, alkyloxy or —OCH₂OCH₂CH₂OCH₃, and R²¹ is hydroxy, —OCN, alkyloxy, heteroaryloxy optionally having suitable substituent, —OCH₂OCH₂CH₂OCH₃, protected hydroxy, chloro, bromo, iodo, aminooxalyloxy, azide, p-tolyloxythiocarbonyloxy, or R²⁵R²⁶CHCOO— (wherein R²⁵ is hydroxy optionally protected where desired or protected amino, and R²⁶ is hydrogen atom or methyl), or R²⁰ and R²¹ in combination form

an oxygen atom of epoxide ring, or

(c) cyclopentyl wherein cyclopentyl is substituted by methoxymethyl, optionally protected hydroxymethyl where desired, acyloxymethyl (wherein acyl moiety is optionally quaternized dimethylamino or optionally esterified carboxy), one or more optionally protected amino and/or hydroxy, or aminooxalyloxymethyl. Preferable examples include 2-formylcyclopentyl.

The definition of each symbol used in the formula (I), specific examples thereof and preferable embodiments thereof will be explained in detail in the following.

“Lower” generally means a group having from about 1 to about 6 carbon atoms unless otherwise indicated.

Preferable examples of the alkyl moiety of “alkyl” and “alkyloxy” include linear or branched aliphatic hydrocarbon residue, such as lower alkyl (e.g., methyl, ethyl, propyl, isopropyl, butyl, isobutyl, pentyl, neopentyl, hexyl and the like).

Preferable examples of “alkenyl” include linear or branched aliphatic hydrocarbon residue having one double bond, such as lower alkenyl (e.g., vinyl, propenyl (e.g., allyl and the like), butenyl, methylpropenyl, pentenyl, hexenyl and the like).

Preferable examples of “aryl” include phenyl, tolyl, xylyl, cumenyl, mesityl, naphthyl and the like.

Preferable examples of the protective group for “protected hydroxy” and “protected amino” include 1-(lower alkylthio)(lower)alkyl such as lower alkylthiomethyl (e.g., methylthiomethyl, ethylthiomethyl, propylthiomethyl, isopropylthiomethyl, butylthiomethyl, isobutylthiomethyl, hexylthiomethyl and the like), with more preference given to C₁-C₄ alkylthiomethyl and most preference given to methylthiomethyl; tri-substituted silyl such as tri(lower)alkylsilyl (e.g., trimethylsilyl, triethylsilyl, tributylsilyl, tert-butyl dimethylsilyl, tri-tert-butylsilyl and the like), and lower alkyldiarylsilyl (e.g., methyldiphenylsilyl, ethyldiphenylsilyl, propyldiphenylsilyl, tert-butyldiphenylsilyl and the like), with more preference given to tri(C₁-C₄) alkylsilyl and C₁-C₄ alkyldiphenylsilyl, and most prefererence given to tert-butyl-dimethylsilyl and tert-butyldiphenylsilyl; acyl such as aliphatic acyl, aromatic acyl and aliphatic acyl substituted by aromatic group, which are derived from carboxylic acid, sulfonic acid and carbamic acid; and the like.

The aliphatic acyl is exemplified by lower alkanoyl optionally having one or more suitable substituent(s) (e.g., carboxy) such as formyl, acetyl, propionyl, butyryl, isobutyryl, valeryl, isovaleryl, pivaloyl, hexanoyl, carboxyacetyl, carboxypropionyl, carboxybutyryl, carboxyhexanoyl and the like; cyclo(lower)alkyloxy(lower)alkanoyl optionally having one or more suitable substituent(s) (e.g., lower alkyl) such as cyclopropyloxyacetyl, cyclobutyloxypropionyl, cycloheptyloxybutyryl, mentyloxyacetyl, mentyloxypropionyl, mentyloxybutyryl, mentyloxypentanoyl, mentyloxyhexanoyl and the like; camphorsulfonyl; lower alkylcarbamoyl having one or more suitable substituent(s) such as carboxy or protected carboxy and the like, such as carboxy(lower)alkylcarbamoyl (e.g., carboxymethylcarbamoyl, carboxyethylcarbamoyl, carboxypropylcarbamoyl, carboxybutylcarbamoyl, carboxypentylcarbamoyl, carboxyhexylcarbamoyl) and tri(lower)alkylsilyl(lower)alkyloxycarbonyl(lower)alkylcarbamoyl (e.g., trimethylsilylmethoxycarbonylethylcarbamoyl, trimethylsilylethoxycarbonylpropylcarbamoyl, triethylsilylethoxycarbonylpropylcarbamoyl, tert-butyl dimethylsilylethoxycarbonylpropylcarbamoyl, trimethylsilylpropoxycarbonylbutylcarbamoyl).

The aromatic acyl is exemplified by aroyl optionally having one or more suitable substituent(s) (e.g., nitro), such as benzoyl, toluoyl, xyloyl, naphthoyl, nitrobenzoyl, dinitrobenzoyl, nitronaphthoyl and the like and arenesulfonyl optionally having one or more suitable substituent(s) (e.g., halogen), such as benzenesulfonyl, toluenesulfonyl, xylenesulfonyl, naphthalenesulfonyl, fluorobenzenesulfonyl, chlorobenzenesulfonyl, bromobenzenesulfonyl, iodobenzenesulfonyl and the like.

The aliphatic acyl substituted by aromatic group may be, for example, ar(lower)alkanoyl optionally having one or more suitable substituent(s) (e.g., lower alkyloxy or trihalo(lower)alkyl and the like), wherein specific examples are phenylacetyl, phenylpropionyl, phenylbutyryl, 2-trifluoromethyl-2-methoxy-2-phenylacetyl, 2-ethyl-2-trifluoromethyl-2-phenylacetyl, 2-trifluoromethyl-2-propoxy-2-phenylacetyl and the like.

Of the above-mentiond acyl, more preferable acyl includes C₁-C₄ alkanoyl optionally having carboxy, cyclo (C₅-C₆)alkyloxy(C₁-C₄)alkanoyl having two (C₁-C₄)alkyl in the cycloalkyl moiety, camphorsulfonyl, carboxy (C₁-C₄)alkylcarbamoyl, tri(C₁-C₄)alkylsilyl(C₁-C₄)alkyloxycarbonyl(C₁-C₄)alkylcarbamoyl, benzoyl optionally having one or two nitro groups, and benzenesulfonyl having halogen, phenyl(C₁-C₄)alkanoyl having C₁-C₄ alkyloxy and trihalo(C₁-C₄)alkyl. Of these, most preferred are acetyl, carboxypropionyl, mentyloxyacetyl, camphorsulfonyl, benzoyl, nitrobenzoyl, dinitrobenzoyl, iodobenzenesulfonyl, 2-trifluoromethyl-2-methoxy-2-phenylacetyl and the like. Preferable examples of the “heterocyclic group consisting of saturated or unsaturated 5 or 6-membered ring having nitrogen atom, sulfur atom and/or oxygen atom” are pyrolyl, tetrahydrofuryl and the like.

The “heteroaryl optionally having a suitable substituent moiety” of the “heteroaryloxy optionally having a suitable substituent” is that exemplified for R¹ of the compound of the formula I of EP-A-532,088, with preference given to 1-hydroxyethylindol-5-yl. The disclosure is incorporated hereinto by reference.

The tricyclo compound (I) used in the present invention is described in the publications EP-A-184162, EP-A-323042, EP-A-423714, EP-A-427680, EP-A-465426, EP-A-480623, EP-A-532088, EP-A-532089, EP-A-569337, EP-A-626385, WO89/05303, WO93/05058, WO96/31514, WO91/13889, WO91/19495, WO93/5059 and the like. The disclosures of these publications are incorporated herein by reference.

In particular, the compounds called FR900506 (FK506), FR900520 (Ascomycin), FR900523 and FR900525 are produced by the genus Streptomyces, such as Streptomyces tsukubaensis, No. 9993 (depository National Institute of Advanced Industrial Science and Technology, International Patent Organism Depositary, Central 6, 1-1, Higashi 1-chome, Tsukuba-shi, Ibaraki-ken, Japan (formerly Fermentation Research Institute, Agency of Industrial Science and Technology, the Ministry of International Trade and Industry), date of deposit: Oct. 5, 1984, deposit number FERM BP-927) or Streptomyces hygroscopicus subsp. Yakushimaensis, No. 7238 (depository National Institute of Advanced Industrial Science and Technology, International Patent Organism Depositary, Central 6, 1-1, Higashi 1-chome, Tsukuba-shi, Ibaraki-ken, Japan (formerly Fermentation Research Institute, Agency of Industrial Science and Technology, the Ministry of International Trade and Industry), date of deposit Jan. 12, 1985, deposit number: FERM BP-928 (EP-A-0184162)), and the compound of the following formula, FK506 (generic name: Tacrolimus) is a representative compound.

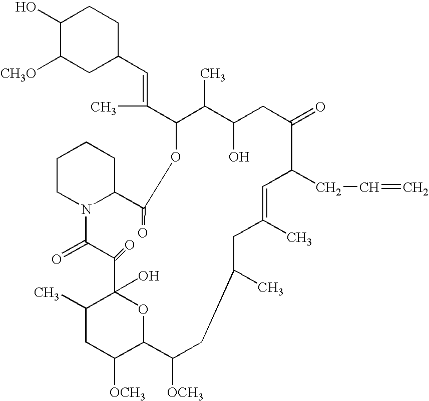

Chemical name: 17-allyl-1,14-dihydroxy-[2-[2-(4-hydroxy-3-methoxycyclohexyl)-1-methylvinyl]-23,25-dimethoxy-13,19,21,27-tetramethyl-11,28-dioxa-4-azatricyclo[22.3.1.0^{4,9}]octacos-18-ene-2,3,10,16-tetraone

Of the tricyclo compounds (I), more preferred is a compound wherein adjacent pairs of R³ and R⁴, and R⁵ and R⁶ each independently form another bond optionally between carbon atoms binding with the members of said pairs;

R⁸ and R²³ each independently show hydrogen atom;

R⁹ is hydroxy;

R¹⁰ is methyl, ethyl, propyl or allyl;

X is (hydrogen atom, hydrogen atom) or oxo;

Y is oxo;

R¹⁴, R¹⁵, r¹⁶, R¹⁷, R¹⁸, R¹⁹ and R²² each independently show methyl;

R²⁴ is 3-R²⁰-4-R²¹-cyclohexyl, wherein R²⁰ is hydroxy, alkyloxy or —OCH₂OCH₂CH₂OCH₃, and R²¹ is hydroxy, —OCN, alkyloxy, heteroaryloxy optionally having suitable substituent, —OCH₂OCH₂CH₂OCH₃, protected hydroxy, chloro, bromo, iodo, aminooxalyloxy, azide, p-tolyloxythiocarbonyloxy or R²⁵R²⁶CHCOO— (wherein R²⁵ is optionally protected hydroxy as desired, or protected amino, and R²⁶ is hydrogen atom or methyl), or R²⁰ and R²¹ in combination form an oxygen atom of epoxide ring; and

n is 1 or 2.

Particularly preferable tricyclo macrolide compounds (I) include, besides FK506, Ascomycin derivatives such as halogenated derivative of 33-epi-chloro-33-desoxy Ascomycin described in Example 66a of EP-A-427,680 and the like.

Other preferable macrolide compounds include Rapamycin described in MERCK INDEX, 12 edition, No. 8288 and derivatives thereof. Preferable examples thereof include O-substituted derivative described at page 1 of WO95/16691, formula A, wherein the 40^{th} hydroxy is —OR₁ (wherein R₁ is hydroxyalkyl, hydroalkyloxyalkyl, acylaminoalkyl and aminoalkyl), such as 40-O-(2-hydroxy)ethyl Rapamycin, 40-O-(3-hydroxy)propyl Rapamycin, 40-O-[2-(2-hydroxy)ethoxy]ethyl Rapamycin and 40-O-(2-acetaminoethyl)-Rapamycin. These O-substituted derivatives can be produced by reacting, under appropriate conditions, Rapamycin (or dihydro or deoxo Rapamycin) and an organic radical bound with leaving group (e.g., RX wherein R is an organic radical desirable as O-substituent, such as alkyl, allyl and benzyl moiety, and X is a leaving group such as CCl₃C(NH)O and CF₃SO₃)). The conditions are: when X is CCl₃C(NH)O, acidic or neutral conditions, such as in the presence of trifluoromethanesulfonic acid, camphorsulfonic acid, p-toluenesulfonic acid or their corresponding pyridinium or substituted pyridinium salt, and when X is CF₃SO₃, in the presence of a base such as pyridine, substituted pyridine, diisopropylethylamine and pentamethylpiperidine. The most preferable Rapamycin derivative is 40-O-(2-hydroxy)ethyl Rapamycin as disclosed in WO94/09010, which is hereby incorporated into the specification by reference.

The pharmaceutically acceptable salt of tricyclo compound (I), Rapamycin and derivatives thereof are nontoxic and pharmaceutically acceptable conventional salts, which are exemplified by salts with inorganic or organic base such as alkali metal salt (e.g., sodium salt, potassium salt and the like), alkaline earth metal salt (e.g., calcium salt, magnesium salt and the like), ammonium salt, and amine salt (e.g., triethylamine salt, N-benzyl-N-methylamine salt and the like).

The macrolide compound of the invention comprises one or more pairs of stereoisomers, such as optical isomers and geometric isomers, which may be included due to conformers or asymmetric carbon atoms and double bonds. Such conformers and isomers are also encompassed in the present invention. In addition, macrolide compounds can form solvates, which also are encompassed by the present invention. Preferable solvates include hydrates and ethanolates.

The instant macrolide compounds and their pharmaceutically acceptable salts are nontoxic. Pharmaceutically acceptable conventional salts may have an inorganic or organic base, such as alkali metal salt (e.g., sodium salt, potassium salt and the like), alkaline earth metal salt (e.g., calcium salt, magnesium salt and the like), ammonium salt, and amine salt (e.g., triethylamine salt, N-benzyl-N-methylamine salt and the like).

As used herein, unless otherwise specifically noted, the term “-macrolide” or reference to a particular macrolide is meant to include all pharmaceutically acceptable salts thereof.

Ophthalmic Compositions

While the present macrolide compounds may be administered in any number of ways, the most convenient forms are contemplated to be eye drops and ointments, which may be prepared according to conventional methods. The optimal concentration of the macrolide compounds is in the range of about 0.01% to about 0.1% (more strictly, 0.01% to 0.1%), more preferably is about 0.03% to about 0.06% (more strictly, 0.03% to 0.06%), with 0.03% being most preferred.

Eye drops, for instance, may be prepared by dissolving the active ingredient in a sterile aqueous solution such as physiological saline, buffering solution, etc., or by providing a powdered composition that is dissolved before use. Eye drops such as the ones as described in EP-A-0406791 (which is incorporated by reference in its entirety) are preferred. Conventional eye drop additives can be used. Such additives include isotonizing agents (e.g., sodium chloride, etc.), buffer agents (e.g., boric acid, sodium monohydrogen phosphate, sodium dihydrogen phosphate, etc.), preservatives (e.g., benzalkonium chloride, benzethonium chloride, chlorobutanol, etc.), thickeners (e.g., saccharide such as lactose, mannitol, maltose, etc.; e.g., hyaluronic acid or its salt such as sodium hyaluronate, potassium hyaluronate, etc.; e.g., mucopolysaccharide such as chondroitin sulfate, etc.; e.g., sodium polyacrylate, carboxyvinyl polymer, crosslinked polyacrylate, etc.). Eye drops may also contain polyvinyl alcohol.

Especially, polyvinyl alcohol as additive is preferably used in the eye drop of the present invention.

Ophthalmic ointments may be prepared by mixing the active ingredient with a base according to conventional methods. Examples ointment bases include, but are not limited to, petrolatum, selen 50, Plastibase and macrogol. In order to increase the hydrophilicity, a surface-active agent, like a detergent or other emulsifier, can be added. The same additives used in the eye drops, such as the preservatives, etc. can also be used in an ointment.

The present formulation can further include other pharmacological active ingredients as far as they do not contradict the purpose of the present invention. For instance, the formulation can include a single or multiple macrolide compounds, and may also include one or more antimicrobial agents as active ingredients for the purpose of treating or preventing bacterial infections. In a combination of plural active ingredients, their respective contents may be suitably increased or decreased in consideration of their effects and safety.

The present agent can be formulated as a sterile unit dose type containing no preservatives.

Methods of Treatment

The term “treatment” used herein includes any means of control such as prevention, care, relief of the condition, attenuation of the condition and arrest of progression.

The patient being treated will generally have a history of dry eye, which typically is characterized by a deficiency in the quantity or quality of the lacrimal fluid (tears). More severe cases may include the presence of corneal and/or conjunctival lesions, like superficial punctate keratitis. Thus, the present methods include methods of treating the ocular surface damage and the ocular discomfort associated with dry eye. As used herein, “dry eye” includes, but it is not limited to, the ocular symptoms observed in hypolacrimation, alacrima, xerophthalmia, Sjögren's syndrome, keratoconjunctivitis sicca, Stevens-Johnson syndrome, ocular pemphigoid, marginal blepharitis, diabetes and the like, dry eye observed after cataract surgery, hypolacrimation associated with video display terminal work, dry rooms and the like.

Dry eye may be diagnosed by many standard methods, such as the Schirmer test and measuring tear break-up time, which evaluate the quantity and quality of the lacrimal fluid. It also may be diagnosed by corneal and/or conjunctival staining in order to detect the lesions, such as superficial punctate keratitis (SPK), that are often associated with dry eye. Several such methods are described in more detail below in the Example, but standard methodologies are familiar to those skilled in the art.

The present macrolide-containing compositions, described above, generally are topically administered to the eyes and/or the surrounding skin, such as the eyelids. The amount and frequency of administration can vary according to sex, age and weight of a human, symptoms to be treated, desirable therapeutic effects, administration routes and period for treatment. However, the inventor has found the optimal concentration of macrolide compound in the ophthalmic composition (eye drop, eye ointment) for treating dry eye syndrome and its symptoms to be in the range of about 0.01% to about 0.06% macrolide compound. Concentrations of up to about 0.1% may be used, but generally those are best formulated as an ointment. When considering all factors, concentrations of 0.03% appear to be best suited for treatment. Preferably, the macrolide compounds is formulated as an eye drop and may be administered several times a day per eye, preferably one to six times, more preferably one to four times, several drops per time, preferably one to four drops. It is most preferred to administer one drop three times per day.

Preferred patients are those having a Schirmer score of less than 7 millimeters per five minutes and/or a superficial punctate keratitis (SPK) score of at least 2. The most responsive patients, however, are those having a Schirmer score of less than 5 millimeters per five minutes and/or an SPK score of at least 3.

The present invention will be described in more detail with reference to the following examples, which are not intended to limit the present invention.

### EXAMPLE

Human patients diagnosed as having kerratoconjunctivitis sicca (KCS; dry eye) were treated with eye drops containing either various concentrations of tacrolimus, a macrolide compound according to the invention or placebo. Eye drops were administered 3 times per day for six weeks (42 days). Patients were evaluated using the Schirmer test; corneal fluorescein staining, conjunctival lissamine green staining and an ocular discomfort score.

The standard Schirmer test was performed without anesthesia, using Whatman N° 41 paper. The strip was placed at the junction of the middle and lateral one-third of the lower eyelid. The strip was removed after 5 minutes and the amount of wetting was recorded in millimeters.

Corneal fluorescein staining was performed by instilling a drop of 0.5% fluorescein ophthalmic solution into the cul-de-sac of the eye. Punctate staining over the entire cornea was then evaluated using a slit lamp with a yellow filter. The following scoring system was used to quantify the extent of superficial punctate keratitis (SPK):

| Score | Extent of Staining |
|---|---|
| 0 | Absent |
| 1 | Stained punctations ≦10% |
| 2 | Stained punctations >10% and ≦25% |
| 3 | Stained punctations >25% and ≦50% |
| 4 | Stained punctations >50% |

Conjunctival lissamine green staining was performed by instilling a drop of lissamine green ophthalmic solution into the cul-de-sac of the eye. Following instillation, punctate staining was evaluated for the nasal and temporal interpalpebral conjunctiva using the following scoring system:

| Score | Extent of Staining |
|---|---|
| 0 | 0% |
| 1 | 1-15% |
| 2 | 16-30% |
| 3 | 31-45% |
| 4 | >45% |

Patients were also evaluated based on ratings of their ocular discomfort, which was a composite of scores for burning/stinging, irritation, photophobia, dry eye sensation and foreign body sensation. Scoring was on a five-point scale, as follows:

| Score | Severity |
|---|---|
| 0 | Absent |
| 1 | Mild |
| 2 | Moderate |
| 3 | Severe |
| 4 | Unbearable |

The results of the lissamine green staining at day 42 showed an improvement over baseline in macrolide-treated patients having a Schirmer score of less than 5 millimeters/five minutes and an SPK score of at least 3. While placebo-treated patients showed decreases of 0.6±3.15 units from baseline, patients treated with 0.01% and 0.06% macrolide compound, showed decreases of 1.8±1.47 units and 3.9±2.03 units, respectively.

Likewise, ocular discomfort improved over baseline in the same treated patient population. Whereas at day 42, placebo-treated patients showed a mean ocular discomfort decrease of 3.1±4.34 units, patients treated with 0.01% and 0.06% macrolide compound showed decreases of 6.0±4.43 units and 6.8±3.01 units, respectively.

This application is based on application No. 60/440,388 filed in the United States of America, the contents of which are incorporated hereinto by reference.

What is claimed is:

1. A method of treating a human patient suffering from dry eye, wherein prior to treatment said patient has a Schirmer score of less than or equal to seven millimeters per five minutes, said method comprising administering to the patient an ophthalmic composition containing a macrolide compound.

2. A method according to claim 1, wherein prior to treatment said patient has a Schirmer score of less than or equal to five millimeters per five minutes.

3. A method according to claim 2, wherein said ophthalmic composition contains from about 0.01% to about 0.1% of said macrolide compound.

4. A method according to claim 3, wherein said ophthalmic composition contains from about 0.03% to about 0.06% of said macrolide compound.

5. A method according to claim 4, wherein said ophthalmic composition contains about 0.03% of said macrolide compound.

6. A method according to claim 1, wherein said macrolide compound is FK506.

7. A method according to claim 1, wherein said ophthalmic composition is an eye drop.

8. A method according to claim 7, wherein said eye drop further contains polyvinyl alcohol.

9. A method according to claim 7, wherein said eye drop contains about 0.03% of said macrolide compound.

10. A method according to claim 7, wherein said eye drop is administered from about one to about four times per day.

11. A method according to claim 1, wherein said macrolide compound has the following formula (I) or a pharmaceutically acceptable salt thereof: 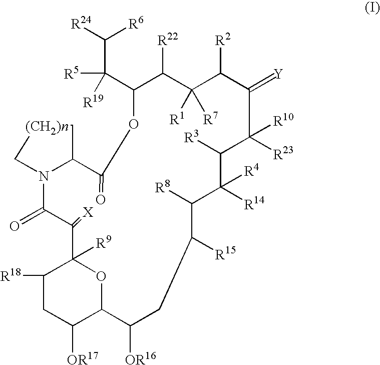
wherein adjacent pairs of R¹ and R², R³ and R⁴, and R⁵ and R⁶ each independently
a) consist of two adjacent hydrogen atoms, wherein R² is optionally alkyl, or
b) form another bond optionally between carbon atoms binding with the members of said pairs;
R⁷ is hydrogen atom, hydroxy, alkyloxy or protected hydroxy, or may form oxo with R¹;
R⁸ and R⁹ each independently show hydrogen atom or hydroxy;
R¹⁰ is hydrogen atom, alkyl, alkyl substituted by one or more hydroxy, alkenyl, alkenyl substituted by one or more hydroxy or alkyl substituted by oxo;
X is oxo, (hydrogen atom, hydroxy), (hydrogen atom, hydrogen atom), or a group of the formula —CH₂O—;
Y is oxo, (hydrogen atom, hydroxy), (hydrogen atom, hydrogen atom), or a group of the formula N—NR¹¹R¹² or N—OR¹³;
R¹¹ and R¹² each independently show hydrogen atom, alkyl, aryl or tosyl;
R¹³, R¹⁴, R¹⁵, R¹⁶, R¹⁷, R¹⁸, R¹⁹, R²² and R²³ each independently show hydrogen atom or alkyl;
R²⁴ is an optionally substituted ring that may contain one or more hetero atom(s); and
n is 1 or 2.

12. A method according to claim 11, wherein said macrolide compound has the following structure:

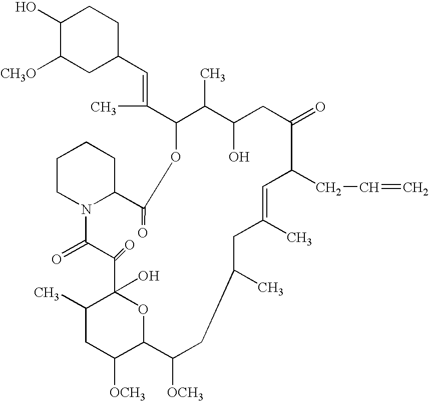

13. A method according to claim 1 or 2, wherein prior to treatment said patient also has a superficial punctate keratitis (SPK) score of at least two.

14. A method according to claim 13, wherein prior to treatment said patient also has a superficial punctate keratitis (SPK) score of at least three.

15. A method of treating a human patient suffering from dry eye, wherein prior to treatment said patient has a superficial punctate keratitis (SPK) score of at least two, said method comprising administering to the patient an ophthalmic composition containing a macrolide compound.

16. A method of treating a human patient suffering from an ocular surface damage associated with dry eye, wherein prior to treatment the patient has a superficial punctate keratitis (SPK) score of at least two, said method comprising administering to the patient an ophthalmic composition containing a macrolide compound.

17. A method of treating a human patient suffering from an ocular discomfort associated with dry eye, wherein prior to treatment the patient has a Schirmer score of less than or equal to seven millimeters per five minutes, said method comprising administering to the patient an ophthalmic composition containing a macrolide compound.

18. A method of treating a human patient suffering from an ocular discomfort associated with dry eye, wherein prior to treatment the patient has a superficial punctate keratitis (SPK) score of at least two, said method comprising administering to the patient an ophthalmic composition containing a macrolide compound.

19. A method of treating a human patient suffering from an ocular surface damage associated with dry eye, wherein prior to treatment the patient has a Schirmer score of less than or equal to seven millimeters per five minutes, said method comprising administering to the patient an ophthalmic composition containing a macrolide compound.
